(19) United States
(12) Patent Application Publication
(10) Pub. No.: US 2022/0024974 A1
(43) Pub. Date: Jan. 27, 2022

(54) METHODS FOR PRODUCING A RICE PROTEIN PEPTIDE AND APPLICATIONS THEREOF

(71) Applicant: WUXI JINNONG BIOTECHNOLOGY CO., LTD, Wuxi City (CN)

(72) Inventors: Tianxiang CHEN, Wuxi City (CN); Qiusheng YU, Wuxi City (CN); Zhenzhen XU, Wuxi City (CN)

(21) Appl. No.: 17/296,555

(22) PCT Filed: Jul. 30, 2019
(86) PCT No.: PCT/CN2019/098294

§ 371 Date: May 25, 2021

(30) Foreign Application Priority Data
Dec. 3, 2018 (CN) 201811463515.X

(51) Int. Cl. C07K 1/34; A61K 8/64; A61Q 19/00; A23L 33/18; A23L 33/185; A23J 3/14; A23J 3/34

(52) U.S. Cl. CPC C07K 1/34; A61K 8/645; A61Q 19/00; A23V 2002/00; A23L 33/185; A23J 3/14; A23J 3/346; A23L 33/18

(57) ABSTRACT

The present invention provides a method for producing a rice protein peptide including (1) preparing a slurry of rice residue protein, followed by sterilizing the rice residue protein; (2) first crushing; (3) undergoing a first proteolysis; (4) second crushing; (5) undergoing a second proteolysis; (6) conducting solid-liquid separation; (7) concentrating; (8) membrane filtering; (9) providing an anti-microbial treatment; (10) performing spray drying. The present method performing super-fine grinding on the proteolytic rice residue protein. During the process of second proteolysis, the protease fully contacts with the substrate, thereby promoting the proteolytic efficiency and taste.

### CROSS-REFERENCE TO RELATED APPLICATIONS

This application is a 371 application of the International Patent Application No. PCT/CN2019/098294 filed on Jul. 30, 2019, which claims priority from the Chinese patent application No. 201811463515.X filed on Dec. 3, 2018, and the disclosures of which are incorporated herein by reference in their entirety.

### COPYRIGHT NOTICE

A portion of the disclosure of this patent document contains material, which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### FIELD OF THE INVENTION

The present invention relates generally to the technical field of rice products. More specifically, it relates to a method for producing a rice protein peptide and applications thereof.

### BACKGROUND

As everyone knows, rice protein peptide is a high-quality plant protein, which has a reasonable and balance composition of essential amino acid, as well as large amounts of methionine and arginine, meeting the ideal model recommended by WHO/FAO. In comparison with other cereal proteins, rice protein has relatively high biological value and protein score, and its nutritional value is comparable to animal protein, such as egg, beef, milk, etc. Rice protein is hypoallergenic, which is very beneficial for infants and young children. The true digestibility, biological value and net protein utilization of the rice protein in the body of a child of pre-primary education is 88.8%, 90.0%, 79.9%, respectively. Such rice protein is very suitable as a kind of nutritional food for infants, young children and specific people. In addition, rice protein has health benefits such as prevention of diabetes, cholesterol, cancer, etc. The rice protein peptide, which is obtained from the proteolysis of rice protein, has various biological activities. For example, it can be anti-oxidant, decrease blood pressure, boost immune system, speed up protein absorption and deposition in the body, promote growth and development and reduce dirt discharged, etc. Therefore, the rice protein peptide can be widely used in food, health products, foods for special medical purposes, beverages and cosmetics.

Rice is the most produced staple food crop in the USA. Besides being eaten directly, rice can also be used as the main raw material applied in starch sugar, pastry, or wine making industries for food deep-processing. In the starch sugar industry, amylase and glucoamylase are often used to hydrolyze the sugar made from rice. After hydrolysis, the rice residue protein is produced, which is a waste generated after sugar production. The main components of the rice residue protein are proteins, residual starch-like substances, and a small amount of fat and cellulose; the protein content of the rice is about 7-8%, while the protein content of the rice residue protein can reach 65%, or higher. Therefore, rice residue protein can be used as an excellent raw material source for preparing rice protein and rice peptides.

In the process of hydrolyzing a rice starch for making the sugar, because the temperature of some processes such as liquefaction is high, leading to protein denaturation and disulfide bond destruction, resulting in poor solubility, water retention and foaming of rice protein. In addition, the molecular weight of rice protein is large, leading to low nutritional value and low value-added of rice protein products, which severely limits the application range of rice protein. There is therefore a need to develop rice protein products with good solubility, water retention and foaming. Due to the specificity and high efficiency of enzymes, proteolysis is currently the main method for preparing rice peptides. However, the existing enzyme technology has some shortcomings such as complicated equipment, high reaction conditions, complicated steps and long reaction cycle, which results in a low yield and a low content of the rice peptides.

Enzymes need to be in full contact with the enzymatic substrate to form a certain spatial structure, and then the substrate is hydrolyzed through proteolysis to produce small molecules of proteins and peptides. A particle size of the material before undergoing proteolysis would directly affect the proteolytic efficiency. When the particle size of the material is larger, the enzyme cannot make sufficient contact with the substrate, causing seriously hindered and affected the reaction efficiency. The rice residue protein is severely denatured during the early processing, which causes the rice residue protein to become very hard, and it is not easy to crush the rice residue protein to below 10 microns by a very extreme approach, thereby affecting the proteolytic efficiency. After undergoing proteolysis for a period of time, the quaternary structure, tertiary structure, even secondary structure and primary structure of the rice residue protein, have changed, making the rice residue protein soft and easy for crushing. Therefore, the present method performing super-fine grinding on the proteolytic rice residue protein. During the process of undergoing a second proteolysis, the protease fully contacts with the substrate, thereby promoting the proteolytic efficiency and taste.

### SUMMARY OF THE INVENTION

To solve the technical problems as above-mentioned, the present invention provides a method for producing a rice protein peptide and applications thereof. The present method performing super-fine grinding on the proteolytic rice residue protein. During the process of undergoing a second proteolysis, the protease fully contacts with the substrate, thereby promoting the proteolytic efficiency and taste.

The present invention provides the following solutions: a method for producing a rice protein peptide comprising:

(1) preparing 5-15% of rice residue protein slurry, followed by sterilizing the rice residue protein slurry at 100-135° C. for a reaction time of 5 seconds to 30 minutes to obtain a material;

(2) crushing the material from step (1) by using a colloid mill or a fine crusher to obtain a crushed rice residue protein, where a D₅₀ particle size distribution of the material is reduced to 30-50 microns;

(3) adding an alkaline protease for undergoing a first proteolysis after adjusting the temperature of the rice residue protein from step (2) to 45-60° C. and the pH value to 9.0-12.0, where the first proteolysis is performed at 45-60° C. for a reaction time of 1-5 hours;

(4) super-fine grinding on the proteolytic rice residue protein by using a micro jet or a ball mill to obtain a first proteolytic fluid, where a D₅₀ particle has a size distribution of the crushed material at 5-10 microns;

(5) adding a neutral protease for undergoing a second proteolysis for a reaction time of 1-5 hours after adjusting the temperature of the first proteolytic fluid from step (4) to 45-60° C. and the pH value to 7.5-6.5, and then performing enzyme deactivation at 85-135° C. for a reaction time of 5 seconds to 30 minutes to obtain a second proteolytic fluid;

(6) conducting solid-liquid separation on the second proteolytic fluid from step (5);

(7) collecting the filtrate from step (6) after completing solid-liquid separation and concentrating the filtrate until the content of a solid content becomes 10-70%, wherein the filtrate is concentrated at 50-90° C. under a vacuum degree of 0.06-0.1 MPa;

(8) conducting membrane filtration of the concentrated solution from step (7) to obtain a feed liquid;

(9) providing an anti-microbial treatment of the feed liquid from step (8), where the condition for the treatment is at 90° C. for a reaction time of 30 minutes;

(10) performing spray drying on the feed liquid from step (9) to obtain a rice protein peptide, where the spray drying conditions include an inlet air temperature at 180° C., an outlet air temperature at 85-90° C., and a water content discharged of no more than 5%.

The protein content of the rice residue protein is 50-90 wt %, and the rice residue protein is derived from the rice residue protein remaining after sugar production and/or starch by-products.

The amount of the alkaline protease as set forth in the step (3) accounts for 0.03-5% of the dry rice residue protein, and the amount of the neutral protease as set forth in the step (5) accounts for 0.03-5% of the dry rice residue protein.

The separation as set forth in the step (6) includes but not limit to vacuum filtration or plate-and-frame filtration; the vacuum degree of the vacuum filtration is 0.1 MPa, and the pressure of the plate and frame filtration is in a range of 0.2-0.7 MPa.

The concentration as set forth in the step (7) includes rotary evaporation or climbing and falling film concentration.

The material of the membrane comprises weak cation exchange column with carboxyl group using chitosan as a pilaster, and the pore size of the membrane is in a range of 0.01-0.2 microns, the pressure is in a range of 0.1-3 MPa, and the operating temperature is in a range of 50−95° C.

There is provided an application of the rice protein peptide prepared according to the method, where the rice protein peptide is processed into healthcare products, foods for special medical purposes, beverages, fruit flavor peptides, energy bars, whey proteins, or meal replacement powder.

There is also provided an application of the rice protein peptide prepared according to said method, where the rice protein peptide is used in cosmetics.

There is also provided an application of the rice protein peptide prepared according to said method, where the rice protein peptide is formulated with one or more peptide selected from soy peptide, collagen peptide, ovalbumin peptide, wheat oligopeptide, corn oligopeptide, pea protein peptide, walnut peptide, peanut peptide, bovine bone peptide, oyster peptide, or a combination thereof to form peptide powder, wherein the rice protein peptide is formulated with or without a sweetener, a filler, or a lubricant accessory.

There is also provided an application of the rice protein peptide prepared according to said method, where the rice protein peptide is used for a peptide tablet or an effervescent tablet with or without magnesium stearate, sodium bicarbonate, citric acid, and silica accessory.

The present invention has the following advantages: The present invention adopts a crushing-1^{st} proteolysis-crushing-2^{nd} proteolysis process, which has high efficiency. The conventional techniques involve crushing the rice residue protein, followed by undergoing proteolysis. The finer the rice protein is crushed, the easier is the proteolysis. However, rice protein is very hard to crush. Normally, when directly crushing the rice protein, the resulting size cannot reach 5-10 microns. However, if an additional proteolysis step is added, the rice protein can be more easily crushed to a very fine size, about 5-10 microns, and it is easier to carry out the 2^{nd} proteolysis as well. Two rounds of crushing and proteolysis can complement each other, leading to higher yield of product.

In comparison with original rice residue protein, the amount of arginine and glutamic acid in the peptide prepared according to the present method is increased, which is beneficial for promoting muscle protein synthesis. In contrast, the amount of the isoleucine and valine is reduced. As the content of amino acids with antagonistic effect is reduced, the nutrients can be absorbed easily, so the nutritional value is high.

In comparison with original rice residue protein, the amount of arginine in the peptide prepared according to the present method is increased, which is beneficial to the rehabilitation of postoperative patients, improving the immunity of patients, and reducing infection rates. In addition, it can improve nitrogen balance in a patient's body, improve the nutritional status and slow down the status of malnutrition, thereby allowing the patient to recover.

The rice protein peptides of the present invention are soluble, where the content of peptides accounts for more than 97.5% of the total protein content; the molecular weight distribution above 80% of rice protein peptides ranges from 1-2000 Da; the protein product yield is greater than 90%; the protein content in the product is higher than 90%, the water content is less than 5%, the fat is <1%, and the ash content is <6%. An index of microorganisms in the present rice protein peptide is qualified. The pH value is in a range of 6.0-7.0.

### BRIEF DESCRIPTION OF THE DRAWINGS

FIG. 1 depicts a flow chat of the overall process of the present invention.

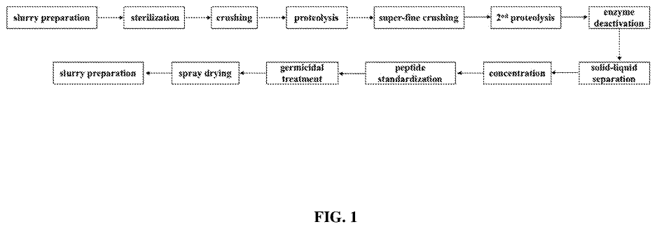

### DETAILED DESCRIPTION

The following description accompanied with examples illustrate the embodiments of the present invention.

### Example 1

There is provided a method for producing a rice protein peptide including the following steps:

(1) preparing 5% of rice residue protein slurry, followed by sterilizing the rice residue protein slurry at 135° C. for a reaction time of 5 seconds to obtain a material;

(2) crushing the material from step (1) to 600 mesh by using a colloid mill to obtain a crushed rice residue protein, where a D₅₀ particle size distribution of the material is reduced to 30 microns;

(3) adding 5% of alkaline protease for undergoing a first proteolysis after adjusting the temperature of the rice residue protein from step (2) to 60° C. and the pH value to 12.0, where the proteolysis process is performed at 60° C. for a reaction time of 5 hours;

(4) performing super-fine grinding on the proteolytic rice residue protein by using a micro jet to obtain a first proteolytic fluid, where a D₅₀ particle size distribution of the crushed material is 5 microns;

(5) adding 5% of neutral protease for undergoing a second proteolysis for a reaction time of 5 hours after adjusting the temperature of the first proteolytic fluid from step (4) to 60° C. and the pH value to 7.5, and then performing enzyme deactivation at 135° C. for a reaction time of 5 seconds to obtain second proteolytic fluid;

(6) conducting solid-liquid separation on the second proteolytic fluid from step (5) through vacuum filtration, where the vacuum degree is 0.1 MPa;

(7) collecting the filtrate part from step (6) after completing solid-liquid separation and concentrating the filtrate until the content of a solid content becomes 70%, wherein the filtrate is concentrated at 90° C. and having a vacuum degree of 0.06 MPa;

(8) conducting membrane filtration of the concentrated solution from step (7) to obtain a feed liquid, where the material of the membrane is a weak cation exchange column with carboxyl group using chitosan as a pilaster. The pore size of the membrane is 0.01 microns, the pressure is 3 MPa, and the operating temperature is at 95° C.;

(9) providing an anti-microbial treatment of the feed liquid from step (8), where the condition for the treatment is at 90° C. for a reaction time of 30 minutes;

(10) performing spray drying on the feed liquid from step (9) to obtain a rice protein peptide, where the spray drying conditions include an inlet air temperature at 180° C., an outlet air temperature at 90° C., and a water content discharged is no more than 5%.

In comparison with the rice residue protein, the amino acid composition in the resulting product of the rice protein peptide has changed. In particular, the amount of the arginine and glutamic acid is increased, which is beneficial for promoting muscle protein synthesis. In contrast, the amount of the isoleucine and valine is reduced. As the content of amino acids with antagonistic effect is reduced, the nutrients can be absorbed easily, so the nutritional value is high, as shown in Table 1 below:

TABLE 1

| Hydrolyzed amino acids | Example 1 | Rice residue protein |
|---|---|---|
| Alanine | 5.12 | 5.36 |
| Serine | 4.94 | 4.38 |
| Leucine | 6.39 | 7.35 |
| Aspartic acid | 9.95 | 7.45 |
| Isoleucine | 3.1 | 3.93 |
| Glycine | 4.32 | 3.7 |
| Arginine | 8.75 | 7.58 |
| Histidine | 1.95 | 2.18 |
| Valine | 4.88 | 5.92 |
| Proline | 3.98 | 4.29 |
| Threonine | 3.52 | 3.22 |
| Phenylalanine | 4.53 | 5.05 |
| Glutamic acid | 18.2 | 16.09 |
| Lysine | 3.08 | 2.98 |
| Tyrosine | 4.21 | 5.04 |
| Tryptophan | 0.86 | 1.08 |
| Cystine | 1.22 | 2.04 |
| Methionine | 1.75 | 2.35 |
| Total | 90.75 | 89.99 |

The rice protein peptides are applied in protein powder, where the formulation contains 30% of rice protein powder, 60% of rice protein peptides, 8% of maltodextrin, and 2% of high-fructose corn syrup.

The rice protein peptides are applied in foods for special medical purposes, where the formulation contains 10 parts by mass of rice protein peptides, 2 parts by mass of maltitol, 0.02 parts by mass of mogroside, 0.01 parts by mass of stevia, 0.001 parts by mass of sucralose, 0.3 parts by mass of citric acid, 0.2 parts by mass of malic acid, 0.04 parts by mass of sodium citrate, 0.005 parts by mass of Vitamin B6, and 0.01 parts by mass of calcium. Such foods for special medical purposes are beneficial to the rehabilitation of postoperative patients, improving the immunity of patients, and reducing infection rates. Also, the foods for special medical purposes can improve nitrogen balance in postoperative patients or those who suffer from a cancer, improve their weight loss and the nutritional status, slow down the status of malnutrition, and reduce length and cost of hospitalization, thereby allowing the patient to recover.

### Example 2

There is provided a method for producing a rice protein peptide including the following steps:

(1) preparing 5% of rice residue protein slurry, followed by sterilizing the rice residue protein slurry at 100° C. for a reaction time of 30 minutes to obtain a material;

(2) crushing the material from step (1) by using a fine crusher to obtain a crushed rice residue protein, where a D₅₀ particle size distribution of the material is reduced to 30 microns;

(3) adding 0.3% of alkaline protease for undergoing a first proteolysis for a reaction time of 1 hour after adjusting the temperature of the rice residue protein from step (2) to 45° C. and the pH value to 9.0;

(4) performing super-fine crushing on the proteolytic rice residue protein by using a ball mill to obtain a first proteolytic fluid, where a D₅₀ particle size distribution of the crushed material is 10 microns;

(5) adding 0.3% of neutral protease for undergoing a second proteolysis for a reaction time of 1 hour after adjusting the temperature of the first proteolytic fluid from step (4) to 45° C. and the pH value to 6.5, and then performing enzyme deactivation at 85° C. for a reaction time of 30 minutes to obtain a second proteolytic fluid;

(6) conducting solid-liquid separation on the second proteolytic fluid from step (5) through plate and frame filtration, where the filter cloth is 750B filter cloth or double-sided thickened polypropylene D60, and the vacuum degree is 0.4 MPa;

(7) collecting the filtrate part from step (6) after completing solid-liquid separation and concentrating the filtrate until the content of a solid content becomes 10%, wherein the filtrate is concentrated at 50° C. and having a vacuum degree of 0.1 MPa;

(8) conducting membrane filtration of the concentrated solution from step (7) to obtain a feed liquid, where the material of the membrane is a weak cation exchange column with carboxyl group using chitosan as a pilaster. The pore size of the membrane is 0.2 microns, the pressure is 0.1 MPa, and the operating temperature is at 50° C.;

(9) providing an anti-microbial treatment of the feed liquid from step (8), where the condition for the treatment is at 90° C. for a reaction time of 30 minutes;

(10) performing spray drying on the feed liquid from step (9) to obtain a rice protein peptide, where the spray drying conditions include an inlet air temperature at 180° C., an outlet air temperature at 85° C., and a water content discharged is no more than 5%.

The physical, and chemical indicators of the resulting product of the rice protein peptide are shown in Table 2.

TABLE 2

| Water content/% | Protein/% | Ash/% | Fat % | Free amino acids/% |
|---|---|---|---|---|
| 3.52 | 91.05 | 4.52 | 0.10 | 0.15 |

The rice protein peptides are applied in cosmetics. For example, the formulation of the toner contains 4% of rice protein peptides, 10% of glycerol, 3% of hyaluronan, 10% of hyaluronic acid, 30% of rose water, 5% of butanediol, 5% of pentanediol, and 33% of pure water.

The cosmetics prepared by the rice protein peptides can resist oxidation, help skin resisting external stimuli, and delay skin aging.

### Example 3

There is provided a method for producing a rice protein peptide including the following steps:

(1) preparing 5% of rice residue protein slurry, followed by sterilizing the rice residue protein slurry at 115° C. for a reaction time of 15 minutes to obtain a material;

(2) crushing the material from step (1) by using a high-speed blender to obtain a crushed rice residue protein, where a D₅₀ particle size distribution of the material is reduced to 40 microns;

(3) adding 1% of alkaline protease for undergoing a first proteolysis after adjusting the temperature of the rice residue protein from step (2) to 55° C. and the pH value to 10.5, where the proteolysis process is performed at 55° C. for a reaction time of 3 hours;

(4) performing super-fine grinding on the proteolytic rice residue protein by using a mechanical impact pulverizer to obtain a first proteolytic fluid, where a D₅₀ particle size distribution of the crushed material is 8 microns;

(5) adding 2% of neutral protease for undergoing a second proteolysis for a reaction time of 4 hours after adjusting the temperature of the first proteolytic fluid from step (4) to 55° C. and the pH value to 7.0, and then performing enzyme deactivation at 100° C. for a reaction time of 15 minutes to obtain a second proteolytic fluid;

(6) conducting solid-liquid separation on the second proteolytic fluid from step (5) through a centrifuge, where the second proteolytic fluid is centrifuged at 4000 rpm/min for 10 minutes;

(7) collecting the filtrate part from step (6) after completing solid-liquid separation and concentrating the filtrate until the content of a solid content becomes 35%, wherein the filtrate is concentrated at 65° C. and having a vacuum degree of 0.8 MPa;

(8) conducting membrane filtration of the concentrated solution from step (7) to obtain a feed liquid, where the material of the membrane is a weak cation exchange column with carboxyl group using chitosan as a pilaster. The pore size of the membrane is 0.1 microns, the pressure is 1 MPa, and the operating temperature is at 80° C.;

(9) providing an anti-microbial treatment of the feed liquid from step (8), where the condition for the treatment is at 90° C. for a reaction time of 30 minutes;

(10) performing spray drying on the feed liquid from step (9) to obtain a rice protein peptide, where the spray drying conditions include an inlet air temperature at 180° C., an outlet air temperature at 87° C., and a water content discharged is no more than 5%.

The molecular weight (Da) distribution of the resulting product of the rice protein peptide is shown in Table 3.

TABLE 3

| Molecular weight | Content/% |
|---|---|
| >10000 | 0.22 |
| 5000-10000 | 2.8 |
| 3000~5000 | 5.38 |
| 2000~3000 | 6.88 |
| 1000~2000 | 16.47 |
| 500~1000 | 22.04 |
| 180~500 | 30.39 |
| <180 | 15.82 |
| Sum of the molecular weight less than 2000 | 84.72 |

The rice protein peptides are applied in composite peptide powder, where the formulation containing 30% of rice protein peptide, 10% of ovalbumin (OVA) peptide, 10% of wheat oligopeptide, 10% of marine fish collagen peptide, 10% of peanut peptide, 10% of soybean peptide, 10% of pea protein polypeptide, and 10% of walnut protein peptide.

The resulting composite peptide powder has more comprehensive nutrition, the amount of amino acids within the peptide powder close to those required in human body and is beneficial for absorption, which can be used as clinical nutrition supplements with dual functions as nutrition and regulation.

The rice protein peptides are applied in effervescent tablets, where the formulation containing 25% of rice protein peptide, 11% of citric acid, 35% of sodium bicarbonate, 8% of sodium carboxymethyl starch, 12% of lactose, 1.5% of aspartame, 1% of sweet orange essence, 4% of polyvinylpyrrolidone (PVP)—K30, 1% of polyethylene glycol, 1% of silicon micropowder, and 0.5% of magnesium stearate.

The characteristics of the resulting effervescent tablets are easy to store and carry, fast disintegration, and convenient to take, which are suitable for the elderly, children and patients with dysphagia. Meanwhile, taking effervescent tablets has some fun as it has a soda-like taste when taken, and tastes better after seasoning, making it more easily acceptable.

What is claimed is:

1. A method for producing a rice protein peptide, comprising:
(1) preparing 5-15% of rice residue protein slurry, followed by sterilizing the rice residue protein slurry at a temperature of 100-135° C. for a reaction time of 5 seconds to 30 minutes to obtain a material;
(2) crushing the material by using a colloid mill or a fine crusher to obtain a crushed rice residue protein, wherein a D₅₀ particle size distribution of the material is reduced to 30-50 microns;
(3) adding an alkaline protease for undergoing a first proteolysis after adjusting the temperature of the rice residue protein to 45-60° C. and the pH value to a range from 9.0-12.0, wherein the first proteolysis is performed at a temperature of 45-60° C. for a reaction time of 1-5 hours;
(4) performing super-fine grinding on the proteolytic rice residue protein by using a micro jet or a ball mill to obtain a first proteolytic fluid, wherein a Dso particle size distribution of the crushed material is 5-10 microns;
(5) adding a neutral protease for undergoing a second proteolysis for a reaction time of 1-5 hours after adjusting the temperature of the first proteolytic fluid to 45-60° C. and the pH value to a range from 7.5-6.5, and then performing enzyme deactivation for a reaction time of 5 seconds to 30 minutes to obtain a second proteolytic fluid;
(6) conducting solid-liquid separation on the second proteolytic fluid;
(7) collecting filtrate after completing said solid-liquid separation and concentrating the filtrate until reaching a solid content of 10-70%, wherein the filtrate is concentrated at a temperature of 50-90° C. under a vacuum degree of 0.06-0.1 MPa;
(8) conducting membrane filtration of the concentrated solution to obtain a feed liquid;
(9) providing an anti-microbial treatment of the feed liquid, wherein the condition for the treatment is at a temperature of 90° C. for a reaction time of 30 minutes;
(10) performing spray drying on the feed liquid to obtain a rice protein peptide, wherein the spray drying conditions include an inlet air temperature of 180° C., an outlet air temperature of 85-90° C., and a water content discharged is no more than 5%.

2. The method according to claim 1, wherein the protein content of the rice residue protein is from 50-90 wt %, and wherein the rice residue protein is derived from the rice residue protein remaining after sugar production and/or starch by-products.

3. The method according to claim 1, wherein the amount of the alkaline protease in the step (3) accounts for 0.03-5% of the dry rice residue protein, and wherein the amount of the neutral protease as set forth in the step (5) accounts for 0.03-5% of the dry rice residue protein.

4. The method according to claim 1, wherein said separation in the step (6) includes vacuum filtration or plate-and-frame filtration, wherein the vacuum degree of the vacuum filtration is 0.1 MPa, and wherein the pressure of the plate-and-frame filtration is in a range of 0.2-0.7 MPa.

5. The method according to claim 1, wherein said concentration in the step (7) comprises rotary evaporation or climbing and falling film concentration.

6. The method according to claim 1, wherein the material of the membrane comprises weak cation exchange column with carboxyl group using chitosan as a pilaster, and wherein the pore size of the membrane is in a range of 0.01-0.2 microns, the pressure is in a range of 0.1-3 MPa, and the operating temperature is in a range of 50-95° C.

7. A method of applying a rice protein peptide prepared according to the method of claim 1 in food processing, comprising processing the rice protein peptide into health products, foods for special medical purposes, beverages, fruit flavor peptides, energy bars, whey proteins, or meal replacement powder.

8. A method of applying a rice protein peptide prepared according to the method of claim 1 in cosmetics.

9. A method of applying a rice protein peptide prepared according to the method of claim 1 in formulating peptide, comprising formulating one or more peptides selected from soy peptide, collagen peptide, ovalbumin peptide, wheat oligopeptide, corn oligopeptide, pea protein peptide, walnut peptide, peanut peptide, bovine bone peptide, oyster peptide, or a combination thereof to form peptide powder, wherein the formulating of the peptide is with or without a sweetener, a filler, and/or a lubricant accessory.

10. A method of applying a rice protein peptide prepared according to the method of claim 1, wherein the rice protein peptide is used for a peptide tablet or an effervescent tablet with or without magnesium stearate, sodium bicarbonate, citric acid, and/or silica accessory.
